(19) United States
(12) Patent Application Publication
(10) Pub. No.: US 2026/0224374 A1
(43) Pub. Date: Aug. 6, 2026

(54) ORTHOPAEDIC JOINT DEVICE, AND METHOD FOR PRODUCING SAME

(71) Applicant: OTTO BOCK HEALTHCARE PRODUCTS GMBH, Wien (AT)

(72) Inventor: Dirk SEIFERT, Wien (AT)

(21) Appl. No.: 19/143,785

(22) PCT Filed: Dec. 18, 2023
(86) PCT No.: PCT/EP2023/086364

§ 371 Date: Jun. 26, 2025

(30) Foreign Application Priority Data
Dec. 28, 2022 (DE) 10 2022 134 918.7

(51) Int. Cl. A61F 2/72; A61F 2/60; A61F 2/64; A61F 2/66; A61F 2/70

(52) U.S. Cl. CPC A61F 2/72; A61F 2/605; A61F 2/64; A61F 2/6607; A61F 2002/701

(57) ABSTRACT

The invention relates to a method for controlling an orthopaedic joint device comprising: an upper part (10) and a lower part (20), which are articulatedly mounted on one another so as to be pivotable about a pivot axis (15); comprising an actuator (30), which is coupled to the upper part (10) and the lower part (20) and influences a pivoting movement of the upper part (10) relative to the lower part (20), wherein the actuator (30) is coupled to a control device (40) which is coupled to at least one sensor (50) for capturing state data relating to the joint device and activates, deactivates or modulates the actuator (30) on the basis of sensor values from the at least one sensor (50); and comprising at least one bio-signal sensor (60), which detects muscle activity or activation of at least one muscle as a bio-signal and transmits it to the control device (40), wherein the actuator is activated, deactivated or modulated on the basis of the bio-signal or bio-signals, wherein the resistance to the pivoting movement against flexion is increased by the actuator (30) on the basis of at least one bio-signal and subsequently the increased resistance against flexion is lowered on the basis of sensor values.

The invention relates to a method for controlling an orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor and comprising at least one biosignal sensor, which records a muscle activity or a stimulation of at least one muscle as a biosignal and transmits it to the control device, the actuator being activated, deactivated or modulated on the basis of the biosignals. The invention also relates to an orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor and comprising at least one biosignal sensor, which records biosignals and transmits them to the control device, the actuator being activated, deactivated or modulated on the basis of the biosignals.

The orthopedic joint device is formed in particular as a prosthesis, orthosis or exoskeleton of the upper or lower extremity, in particular for artificial ankle joints, artificial knee joints, artificial hip joints, artificial shoulder joints or an artificial elbow joint.

Orthopedic joint devices such as prostheses, orthoses or exoskeletons allow pivoting of at least two components about a pivot axis, so that an upper part can perform a pivoting movement relative to a lower part about this pivot axis. In the case of a prosthesis, the upper part of the joint may be coupled to a prosthesis socket or to an endo-exo implant in order to secure a lower part and optionally further prosthetic components to a limb stump. In the case of a prosthesis of a lower extremity, the socket is formed for example as a femoral socket which is coupled to an upper part of a prosthetic knee joint and on which a shank part comprising a prosthetic foot is pivotably mounted. Arranged between the upper part and the lower part, there is an actuator with which the relative pivoting movement can be influenced. In the case of passive actuators, the pivoting movement is influenced or varied by a resistance being presented against the pivoting movement. The resistance can be varied in order to be able to generate an adapted pivoting behavior. The adaptation takes place for example on the basis of sensor data, by particular loads, accelerations or movements, or alternatively states. Passive actuators are in particular pneumatic or hydraulic resistances, magnetorheological resistances or other braking devices, via which kinetic energy is converted in the heat energy. There are furthermore active actuators, with which the pivoting movement of the upper part relative to the lower part can likewise be influenced. Drives, for example motors or force accumulators, may act as a brake in generator operation or charging operation. It is likewise possible to influence a pivoting movement via an active actuator to the extent that pivoting takes place from a neutral setting or a pivoting movement is reinforced. By the activation of the actuator, a pivoting movement may also be opposed without reversing it. An active actuator then likewise acts as a brake and modulates the pivoting movement.

Modern controls provide for the pivoting movement being influenced via the actuator on the basis of sensor values, because the sensors record state data or movement data of the joint device. The actuator is activated, deactivated or modulated on the basis of sensor values, for example in order to provide a corresponding resistance after a particular setting or a particular joint angle is reached or under a particular load, or to initiate or reinforce pivoting. Actuators are furthermore activated, deactivated or modulated due to biosignals. Biosignals are signals which are recorded via at least one biosignal sensor and are transmitted to the control device. Biosignals are in particular signals which recognize or represent a muscle activity, the stimulation of a musculature or the intention of the stimulation[SK1], for example by mechanical recording of variations by means of a pressure cuff or the like, by electromagnetic sensors which record myoelectrical signals or nerve signals, or by electromechanical sensors which record the activities of a patient or user of the orthopedic joint device via ultrasound or length changes. The sensors for the direct or indirect recording of muscle activities or of the activation of the musculature are the biosignal sensors. On the basis of the biosignals which are transmitted to the control device, the actuator is activated, deactivated or modulated in order to influence the pivoting movement.

Particular ways of influencing of the orthopedic joint device on the basis of sensor data require of the user of the orthopedic joint device that particular states or positions are maintained over a prolonged length of time. If a joint device is intended to be blocked in a particular position, for example in order to exert a standing function or a holding function, the corresponding orthopedic device must be kept motionless or almost motionless for a particular length of time in a particular setting. The control then recognizes that no further movement is to be expected and then locks the joint device. This may be elaborate and difficult.

It is an object of the present invention to provide an orthopedic joint device and a method for its control, with which the use of the orthopedic joint device can be facilitated and made more reliable for the patient.

According to the invention, this object is achieved by a method having the features of the main claim and by an orthopedic device having the features of the further independent claim. Advantageous embodiments and developments of the invention are disclosed in the dependent claims, the description and the figures.

The method for controlling an orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor, and comprising at least one biosignal sensor, which records a muscle activity or a stimulation of at least one muscle as a biosignal and transmits it to the control device, the actuator being activated, deactivated or modulated on the basis of the biosignal or the biosignals, provides that the influence on the pivoting movement by the actuator against a flexion, thus a change of the flexion resistance, is increased on the basis of at least one biosignal, and the increased resistance against a flexion is subsequently reduced on the basis of sensor values. The increase of the flexion resistance, i.e. the strengthened influencing of the pivoting movement against a flexion by the actuator, takes place on the basis of a biosignal, for example via a detection of a contraction, of a cocontraction or by picking up nerve signals or electromechanically by recording a length change, for example of an adhesively bonded sensor or a liner. For example, it is therefore possible to lock a joint by a contraction or cocontraction. In the absence of the biosignal, for example by a reduction of the muscle tension, however, a reduction of the flexion resistance does not take place, or at least does not necessarily take place, rather the increased resistance against the flexion is reduced on the basis of sensor values, that is to say sensor values which are formed and intended for the recording of state data of the joint device.

In one embodiment, the increased flexion resistance is reduced only on the basis of sensor values or on the basis of a combination of sensor values and at least one biosignal. Even in the case of continued activation of the musculature, the flexion resistance may therefore be reduced and a flexion may optionally be reinforced only in the presence of corresponding state data such as acceleration, angular settings, orientations in space, relative or absolute positions, attitude change or loads, optionally in combination with a corresponding biosignal. Loads may be forces, moments, pressures, force action points and/or lever arms. The time derivatives and profiles of state data may also be employed for the control.

The sensors in one embodiment detect, in particular, a movement of the upper part or of the lower part absolutely or relative to one another.

In one embodiment, the increased flexion resistance is maintained, independently of a variation of the biosignal, until at least one sensor signal due to a movement or state change of a defined size is detected by the at least one sensor. A threshold value is therefore established for triggering a reduction of the flexion resistance, the reduction being linked with reaching or exceeding or falling below this threshold value. For example, the upper part and/or the lower part must be moved, for example pivoted, with a particular speed before the flexion resistance is reduced again. Particular load limits may also have to be exceeded or fallen below so that the reduction of the flexion resistance is triggered. It is also possible that a plurality of limit values are fallen below or exceeded. Moreover, auxiliary variables may be calculated from a plurality of input signals, these in turn being compared with one or more threshold values or limit values and triggering a reduction of the flexion resistance. Besides control via limit values, the flexion resistance may also be adapted continuously and/or in a plurality of discrete stages based on the sensor values. Alternatively or in addition, algorithms of signal processing, statistics, classification, machine learning and/or artificial intelligence may be used for the control by means of biosignals and/or sensors. Model-based methods may also be used for the control.

In one embodiment, the at least one biosignal triggers the increase of the flexion resistance independently of the state of the orthopedic joint device, in particular independently of the positions, load and/or a movement of the joint device or of the upper part or of the lower part. A flexion block or an increase of the flexion resistance may therefore be carried out in every state of the orthopedic joint device. This is advantageous in particular in order to bring about rapid locking or a rapid increase of the flexion resistance in an emergency situation. One natural reflex in unforeseen situations, for example when stumbling, is tensing of muscles, and in particular also a cocontraction. A strong biosignal is emitted by such an involuntary cocontraction, which may be evaluated as emergency locking or an emergency response and always triggers an increase of the flexion resistance.

Alternatively, the biosignal triggers the increase of the flexion resistance only when no movement or state change of the upper part and/or of the lower part, or only one that lies below a limit value, is detected by the at least one sensor. Alternatively or in addition, the biosignal triggers the increase of the flexion resistance only within a certain position range, or absolute and/or relative angle range, for example a certain extent of inclination of the shank relative to the femur or relative to an external reference orientation, for example gravity, or in a certain knee angle range. A further or alternative condition is for example also a load, in particular an axial load against the direction of gravity, of the upper part and/or of the lower part, which must be detected before the flexion resistance increase is carried out. For example, an increase of the flexion resistance or blocking of the orthopedic joint device is therefore enabled only in particular situations, for example when a prosthesis, orthoses or exoskeleton is loaded.

Sensor values are in one embodiment provided with a threshold value or a plurality of threshold values, which must respectively be exceeded or fallen below before the control device carries out a reduction of the flexion resistance after an increase has taken place.

The orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor and comprising at least one biosignal sensor, which records biosignals and transmits them to the control device, the actuator being activated, deactivated or modulated on the basis of the biosignals, provides that the control device is adapted to increase the influence by the actuator against a flexion on the basis of a biosignal and to reduce the increased flexion resistance on the basis of sensor values. The actuator is in one embodiment configured as an active actuator comprising a drive, for example in the form of an electric motor or a force accumulator, or as a passive actuator which acts as a brake or damper.

The joint device may be arranged or formed on a lower or upper extremity, in particular as an artificial ankle joint, as an artificial knee joint, as an artificial hip joint, as an artificial elbow joint or as an artificial shoulder joint.

The at least one sensor for recording state data or movement data of the orthopedic joint device is arranged on the upper part and/or on the lower part and records in particular settings in space, settings of the upper part and lower part with respect to one another, forces, moments, accelerations, speeds or variations of states.

Biosignals are any biologically generated signals which enable deliberate or involuntary driving of the orthopedic joint device. The stimulation of one or more muscles may be deduced from biosignals. The stimulation of one or more muscles may involve the intention to stimulate them, in particular when the musculature to be stimulated is no longer present or can no longer be stimulated, for example by paralysis. A biosignal may be an electrical signal, for example nerve signals of peripheral nerves, electrical signals during contraction of the musculature (EMG, electromyography), but also signals of the central nervous system. Electrical signals may be recorded via lead electrodes. If items of information are encoded in a signal, decoding may initially take place before the signal is employed for the control. A biosignal may be a chemical or electrochemical signal, for example the concentration of a substance, the interaction of molecules or an electrochemical gradient. These variables may for example be determined via interaction with light, for instance absorption, or excitation by means of light and subsequent emission. Biosignals may also be conductivities of tissues and/or body parts. Biosignals may also be mechanical parameters such as force, pressure, length and/or length as well as their variations as a function of time, for example the pulse or the length change of a muscle or of structures inside the muscle. These variables may for example be determined by pressure sensors in a socket or cuffs, which record the thickening of a muscle during its contraction. Length changes and geometry changes may also be determined via ultrasound. Biosignals may be recorded invasively or noninvasively. Invasive sensors are for example implanted myoelectrical electrodes, electrodes placed around a nerve or needle electrodes in the brain or spinal cord. Signals of invasive sensors may communicate out wirelessly in order to avoid crossing the skin. The energy supply may also be enabled wirelessly by means of an inductive energy supply. In the case of osseointegrated supplies by means of an endo-exo implant, the attachment of the sensors may take place via the implant. It is also possible for biosensors to be partially invasive. For example, magnetic bodies may be introduced invasively into the tissue, for example the muscle or a tendon, and the displacement of the magnets with respect to one another during movement or tensioning of the musculature may be recorded by external sensors. Several methods may also be combined, or a variety of biosignals may be recorded, particularly in order to achieve greater robustness in relation to interference and detection errors. In one embodiment, the control of the orthopedic device is based on at least one biosignal that correlates with a physical or intended muscle tensioning, contraction and/or stimulation of the musculoskeletal system, in particular via electromyography. By such a biosignal, the wearer is able to exert a possible influence on the control. Different filters and signal processing algorithms may be applied to the recorded biosignals and the resulting variables may be employed for the control. It is therefore possible, for example, to perform driving via a biosignal only when the biosignal fulfills particular criteria, or has a certain spectrum or a certain time profile. For example, a high-frequency alternating signal may be lowpass-filtered and rectified and this signal may then be employed for the control. Besides the biosignal, computed variables such as time derivatives, integral variables, statistical variables or properties in the frequency range may also be employed for the control.

In one embodiment, the stimulation of a muscle or plurality of muscles is ascertained and employed for the control. In a knee prosthesis or orthosis, for example, a lead electrode may be placed on the front side of the thigh and record the contraction of the quadriceps femoris muscle or the biceps femoris muscle, and this biosignal may be employed for the control. Control via only one muscle is particularly simple and economical. It is also possible that the contraction of a plurality of muscles is recorded but only the contraction of one muscle or some of the muscles is employed for the control. In the case of control via a plurality of muscles, a cocontraction, i.e. the simultaneous tensioning of a plurality of muscles, of two or more muscles may be ascertained and employed for the control. A cocontraction need not necessarily involve the contraction of an agonist and antagonist. It is, however, possible to employ different muscle activations or different muscles and/or muscle groups for the control in different situations, movement phases or modes. In some movement situations, particular muscles are tensioned involuntarily, so that it is no longer possible to deduce due to the muscle activation unequivocally whether an increase of the resistance should take place. Some muscles are also biarticular, for example extending the hip and flexing the knee at the same time, so that a knee extension moment is necessarily also applied in the event of a hip extension. This synergy may be utilized for particularly intuitive control.

In one embodiment, methods of machine learning and/or artificial intelligence are applied to the biosignals and the continuous or discrete items of information obtained therefrom, for instance classes, are employed for the control. By means of methods of pattern recognition, different signal features of a biosignal or plurality of biosignals may be used in order to deduce a specific stimulation, for example a specific activation pattern of a plurality of muscles, or the distinction between deliberate or involuntary muscle tensioning. It is therefore possible to carry out a resistance increase and/or reduction only when there is a specific signal pattern. Algorithms of machine learning and/or artificial intelligence may, alternatively or in addition, also be applied for the variation of the resistances and therefore also to the other sensor values.

In one embodiment, the control by means of the at least one biosignal is dependent on a situation, a movement pattern, a movement phase and/or a mode of the orthopedic device. For example, the control by means of a biosignal may be configured differently in a special mode for riding a bicycle than in a mode for walking. It is also possible for the control to depend on the current movement pattern, for example for a different control to be active when walking a straight line than when walking around a corner, or for a different control to be active when walking quickly than when walking slowly. The control may also be varied as a function of the movement phase. For example, a different control may take place in the terminal stance phase than in the preceding swing phase. The movement phases may be ascertained via the sensors. It is also possible that no control by means of a biosignal is active in specific modes, movement patterns or movement phases. Last but not least, it is possible that the user can temporarily or permanently activate and/or deactivate the control by means of biosignals via interfaces, for instance control elements or an app. The adaptation as a function of the situation, a movement pattern, a movement phase and/or a mode may relate both to the criteria for the resistance increase and/or reduction based on the biosignal and to parameters of the resistance increase and/or reduction, for example a sensitivity or amplification of the resistance adaptation as a function of the biosignal, the extent of the resistance adaptation or the type of resistance adaptation, for instance a change of damping, of a stiffness, of an equilibrium attitude and/or of a moment.

In one embodiment, besides a flexion resistance, at least one extension resistance is also increased on the basis of the at least one biosignal, optionally to the extent of a block. The criteria for the increase and decrease of the extension resistance as well as the influenced parameters of the control may differ from those of the flexion resistance.

In one embodiment, a flexion resistance remains increased during driving via a biosignal. The increase of the flexion resistance in this case refers to the resistance that there would be without driving via a biosignal. In a conventional standing function of a prosthetic knee joint, in which flexion is for example fully blocked when standing, a flexion resistance is for example reduced when the prosthesis is relieved, the knee joint is extended or the leg is rapidly rotated forward or backward. Although this behavior is advantageous in many situations, it may be disadvantageous in others. Accordingly, in one embodiment with driving via the biosignal, the resistance may remain increased or optionally blocked even though a reduction of the flexion resistance would take place on the basis of other sensors. In this case, the biosignal overrules the other sensors.

In one embodiment, the profile of the increase and/or reduction of the flexion resistance is time-controlled and/or limited in rate of change. In particular during reduction of the flexion resistance based on the at least one biosignal and/or other sensors, abruptly reducing the resistance may feel unpleasant, or may even be unsafe. Accordingly, it is advantageous not to increase or reduce the resistance suddenly, but to do so continuously or with a reduced or restricted rate of change even if the underlying biosignals change with a very high rate of change, or abruptly. For example, a time profile for the changes between the increased and normal flexion resistances may be stored in the control and used, or subjected to lowpass filtering.

In one embodiment, the change of the flexion resistance, in particular the extent of the increase and/or reduction of the resistance, also depends on other variables, which are for example recorded via sensors, in particular relative angles and segment angles, loads and/or their time derivatives and/or profiles. For example, the increase of the flexion resistance may depend on a knee angle, a shank angle, a pivoting speed, or on an ankle or knee moment.

The described control methods may also be applied to a foot with a mobile ankle joint or a hip joint. A plurality of axes of a joint may also be controlled, for example both the flexion and extension as well as the adduction and abduction of a hip joint. In the case of a foot, it is above all expedient to augment the resistance against a dorsiflexion for standing. A resistance against a dorsiflexion may be augmented based on one or more biosignals, the dorsiflexion resistance not necessarily being reduced in absence of the biosignal, this rather taking place based on sensor values, i.e. sensor values that are formed in order to record state data of the joint device. In the case of a hip joint, it is above all expedient to augment the resistance against hip flexion for standing. The resistances of a plurality of joints may also be driven simultaneously.

In one embodiment, the flexion resistance is increased via one or more impulses of the at least one biosignal. Only brief driving then takes place via the biosignal, the flexion resistance initially being maintained following cessation of the biosignal and the reduction of the flexion resistance subsequently taking place based on sensor values.

In one embodiment, the flexion resistance in an unloaded state of the orthopedic device is increased based on at least one biosignal. This may be useful in order for example to reposition the leg treated with an artificial knee joint, for example in order to enter an automobile. Without the increased flexion resistance, the knee joint would be flexed by the weight acting on the shank and foot when the leg is lifted forward.

In one embodiment, the flexion resistance increased by the biosignal is reduced again when a certain range of an angular position, a load and/or a speed is exceeded or after a defined time. In the case of a leg prosthesis or orthosis, for safety reasons it is expedient for example to reduce a flexion resistance in a knee joint when a particularly pronounced backward or forward inclination of the leg is recognized or the leg is rotated forward or backward particularly rapidly. In the event of a particularly high load, it may also be expedient for safety reasons to reduce the flexion resistance in order to prevent an overload of the attachment to the body, or of the body per se. In such situations, involuntary driving via the biosignal by the user may occur, which necessitates the reduction via sensor signals.

In one embodiment, the increased flexion resistance is only partially reduced based on sensors in the event of persistent driving via the biosignal. Only when the driving via the biosignal is also reduced does full reduction of the flexion resistance take place to a corresponding resistance level. By such driving, a flexion resistance due to the driving by means of a biosignal may remain partially increased even though a reduction of the resistance to a greater extent would take place based on the other sensors.

The increase of the flexion resistance may start with a high, non-blocking resistance such as is advantageous for a leg prosthesis, for example, for stable upright standing or during the stance phase of walking on different surfaces or surface inclinations. It is also possible for an increase to take place starting with a very low or minimal resistance, such as there is for example in the swing phase of a leg prosthesis or in the case of a relieved leg prosthesis. If the flexion resistance is increased by the driving via a biosignal, a reduction to the initial level may again take place based on sensor data. Alternatively or in addition, a reduction to a minimal flexion resistance may take place. This is advantageous particularly in the case of a leg prosthesis or orthosis in the terminal stance phase or when relieved, in order to enable easy initiation of the swing phase or to actively reinforce a flexion movement. The reduction may take place despite continued activation by the biosignal on the basis of state data of the prosthesis or orthosis, which are ascertained via sensors, for example based on a forward inclination of a shank and a load on the forefoot.

Exemplary embodiments of the invention are explained in more detail below with the aid of the figures, in which:

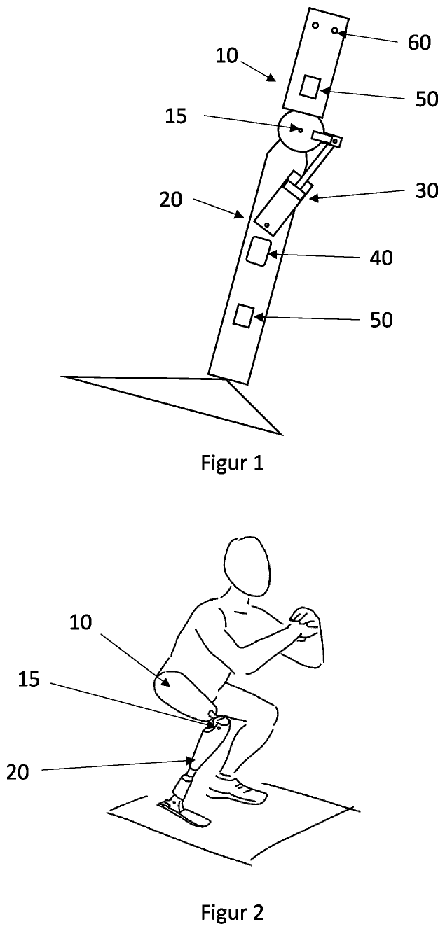

FIG. 1 shows a schematic representation of an orthopedic joint device;

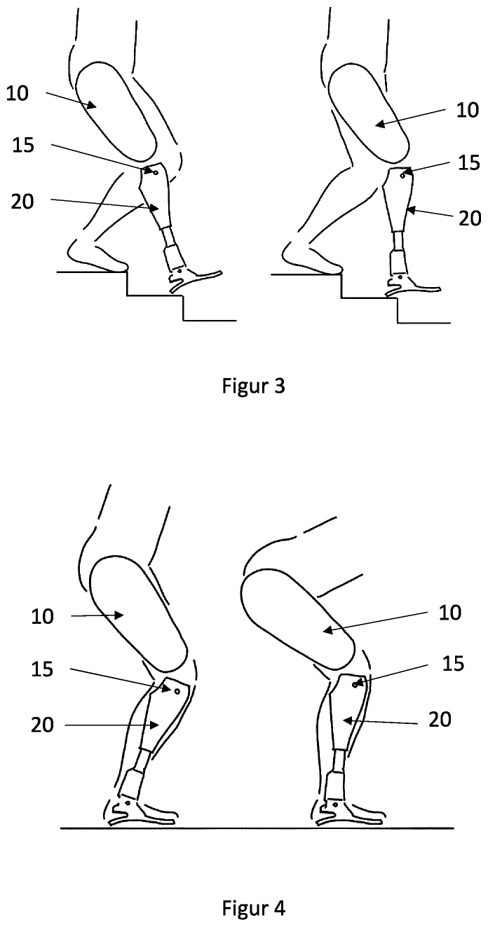

FIGS. 2 to 6 show various working situations;

FIG. 7 shows a schematic representation of the control function;

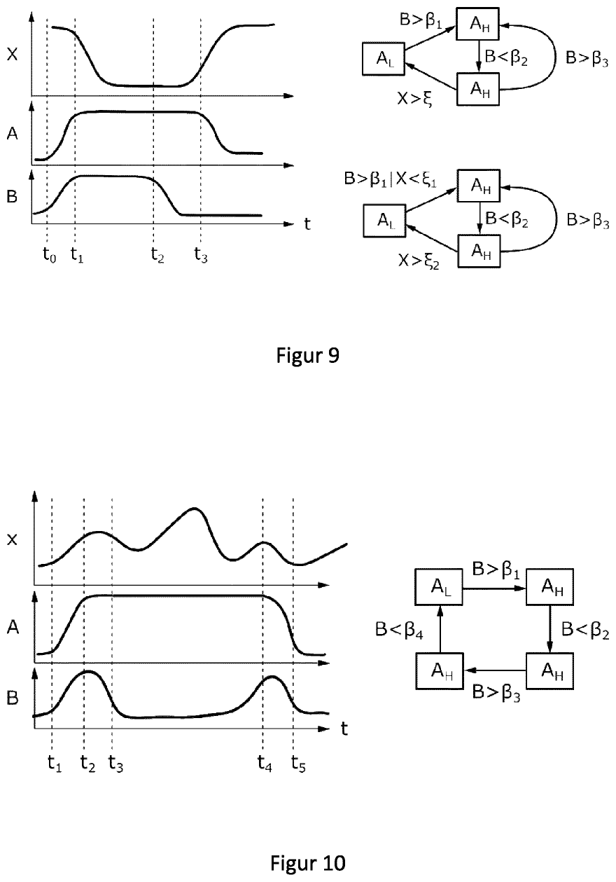

FIGS. 8 to 12 show various control schemes;

FIG. 13 shows correlations between a biosignal and a resistance parameter;

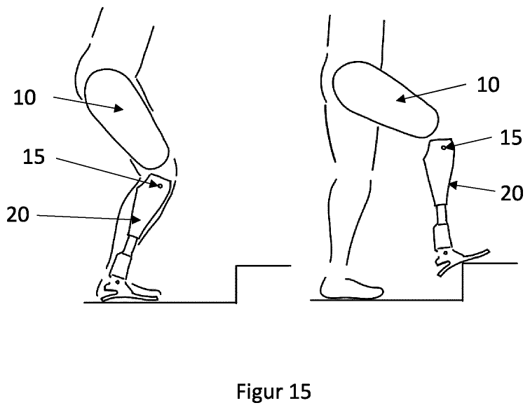

FIG. 14 shows schematic representations of a resistance increase; and

FIG. 15 shows a further working situation.

FIG. 1 is a schematic representation of an orthopedic joint device in the form of a prosthesis of the lower extremity. The orthopedic joint device comprises an upper part 10 and a lower part 20, which are mounted on one another so that they can pivot about a pivot axis 15. In order to influence the relative pivoting of the upper part 10 with respect to the lower part 20 about the pivot axis 15, an actuator 30, which in the exemplary embodiment represented is configured as a hydraulic damper, is arranged on the upper part 10 and on the lower part 20. In alternative embodiments, the actuator 30 may be equipped as an active actuator comprising a drive, for example an electric motor, a force accumulator or another version of the drive, in order to influence a pivoting movement of the upper part 10 relative to the lower part 20. In the exemplary embodiment represented, the actuator 30 is formed as a passive actuator and provides a resistance against a flexion movement and optionally against an extension movement. The actuator 30 is coupled to a control device 40, which in the exemplary embodiment represented is arranged on the lower part 20. The control device 40 is coupled to sensors 50, which are arranged on the upper part 10 and/or on the lower part 20. The sensors 50 record state variables of the joint device, for example positions, attitudes, forces, moments, accelerations or settings of components in space or with respect to one another, in which case a multiplicity of sensors may be used in order to record the desired state variables of the orthopedic device. The sensors 50 are coupled to the control device 40 and transmit corresponding sensor data or sensor signals to the control device 40, ability of the actuator 30 to influence the pivoting movement or pivotability of the upper part 10 relative to the lower part 20 being varied on the basis of the sensor values or sensor signals. For example, the flexion resistance and/or extension resistance are varied as a function of the sensor values. For this purpose, valves or throttles are readjusted. In an alternative embodiment, magnetic fields may be varied in order to vary the viscosity of magnetorheological fluids. In the case of a mechanical braking device, braking forces may be increased or reduced in order to generate an adapted resistance behavior of the actuator 30. In the case of an active drive which comprises at least one electromechanical actuator, resistances against a movement may be applied, or alternatively movements may be reinforced, via currents or voltages. Besides the application of moments and moment profiles, via control algorithms and items of sensor information pertaining to the pivoting movement of the upper part and lower part, trajectories may be tracked or system properties may be emulated in the scope of impedance or admittance regulation. For example, the behavior of a linear or nonlinear spring, the behavior of a damper or an inertia, or a combination of a plurality of properties, may be emulated so that a pivoting movement against a flexion can be influenced. Such driving offers a high degree of flexibility. Pivoting movements may also be actively reinforced by such controls. If a moment or a force is applied via an actuator without pivoting taking place, for example because external and internal forces are in equilibrium, the pivoting movement is likewise countered by a resistance. Via actuators, energy accumulators, for example a hydraulic spring accumulator, may also be activated and deactivated, gear transmission ratios of drives may be varied and/or the latter may be coupled or uncoupled. These types of actuation may likewise be used in order to influence the pivoting movement. Resistances are forces and moments that are applied by actuators in order to influence a pivoting movement. A resistance may be presented against a pivoting movement, but may also be a reinforcement of a pivoting movement, that is to say it may act in the direction of the pivoting.

Formed or fastened on the upper part 10, for example on a prosthesis socket or on a holding device, there are biosignal sensors 60 which are formed in order to record muscle activities or a stimulation of a muscle or the musculature and generate biosignals which are transmitted to the control device 40. The biosignal sensors 60, which may be formed as lead electrodes, are coupled to the control device 40 via a conductive connection or wirelessly and transmit corresponding signals to the control device 40, which activates, deactivates or modulates the actuator 30 likewise on the basis of the biosignals in order to vary the resistance or to influence the movement behavior or the ability of the components to move with respect to one another.

In contrast to the embodiment of the orthopedic joint device as a prosthesis, it may also be formed as an orthosis, in which case, instead of the shank part as the lower part 20, a shank rod is formed on an artificial knee joint. The upper part 10 is then a femoral rod, which may be secured to the thigh via corresponding fastening devices such as belts, shells or cuffs. The biosignal sensors 60 are fastened either separately on the thigh or on another musculature group, and may also be arranged on the fastening device. The orthopedic joint device may also be formed as an exoskeleton as a special form of an orthosis. As an alternative to the represented embodiment as an orthopedic joint device of the lower extremity, the orthopedic joint device may also be formed for an upper extremity, for example as an artificial shoulder joint or artificial elbow joint for a prosthetic arm, or as an orthosis or an exoskeleton for an arm.

The activation, deactivation and optionally the modulation of the actuator in order to influence a pivoting movement or the pivotability of the joint device takes place on the basis of at least one biosignal from the biosignal biosensor, in such a way that the influencing of the pivoting movement or pivotability against a flexion is increased when there is a corresponding biosignal. Influencing the pivoting movement also means that a pivoting movement is blocked so that the lower part 20 is locked relative to the upper part 10 and no relative movement between the two parts takes place or can take place. The influencing of the pivotability of the upper part 10 relative to the lower part 20 may thus consist in blocking the joint in the flexion direction or increasing the flexion resistance. The influencing is carried out by either a passive actuator or an active actuator. In order to reduce the increased flexion resistance of the joint device again, this takes place on the basis of sensor values, i.e. values that are transmitted from the sensor 50 or the sensors 50 to the control device 40, optionally in conjunction with or as a function of a biosignal. Such control of the orthopedic joint device, for example of a lower extremity, is advantageous and expedient for example for a standing function in which a flexion, for example of the knee joint, is intended to be impeded or blocked.

FIG. 2 shows a user of an orthopedic joint device of a lower extremity, comprising an upper part 10, which has a prosthesis socket, and a lower part 20 in the form of a shank part with a prosthetic foot fastened distally thereon. The upper part 10 can pivot relative to the lower part 20 about the pivot axis 15. The actuator, as represented in FIG. 1, is arranged in the lower part 20 and is fastened on the upper part 10 with its upper end. The user of the orthopedic joint device is in a deep squat, in which both legs are loaded. It is known from the prior art that in a bent stationary stance, which may be recognized via recording of the knee angle, the resistance against a flexion is increased until relaxed standing is possible. This takes place for example by blocking the knee joint or by applying an extension moment. In order to signal to the control device that a corresponding function should be activated, the user must exhibit a predefined standard behavior and hold the orthopedic joint device in a particular state, for example adopt a static equilibrium position. For this purpose, an increased extension moment and possibly partial relief of the side treated with the orthopedic joint device are necessary. Adopting such a position is difficult to impossible in many settings, for example in the deep squat represented, under high loading of the treated side or under loading of the forefoot. For the user, it is therefore sometimes difficult at all to satisfy the necessary prerequisites so that the standing function is activated in the conventional way. Particularly in unstable positions, this is difficult, demanding in terms of coordination and strenuous.

According to the proposed method, an activation of the standing functions with an increased resistance against a flexion is recognized via at least one biosignal which is recorded by the biosignal sensor or the biosignal sensors and transmitted to the control device. By the biosignal or plurality of biosignals, the flexion movement may be stopped or slowed in situations in which it is not possible for a user to adopt a necessary equilibrium position for a certain length of time. By the activation or the modulation of the actuator due to at least one biosignal, it is possible sometimes for the first time to adopt the stationary standing position or to facilitate the adoption of the standing position. As soon as the desired position is reached, the increased resistance against a flexion may remain active even without the further presence of at least one biosignal. A user can therefore stand and remain in the represented deep squat substantially more easily.

FIG. 3 represents another working situation of an orthopedic joint device, namely descending a flight of steps. When the prosthetic foot is put down on a lower step, it is particularly difficult to impossible to pause this movement and maintain a stationary standing position, because a high flexion moment is applied about the pivot axis 15 and no stabilization can take place via the prosthetic foot since it is only placed with the heel on the front edge of the lower step. The prosthetic foot rolls over the edge of the step. In this situation, it is not possible or is extraordinarily difficult for a user to activate a standing function in the conventional way. By activation of a biosignal, for example a muscle contraction or a muscle cocontraction, corresponding sensor values are recorded by the biosignal sensors or lead electrodes and transmitted to the control device and an increased flexion resistance is presented against a knee flexion or a further knee flexion, optionally to the extent of blocking the joint. Such control and an increase of the resistance against a flexion may take place dynamically when walking down the flight of steps, in particular when slowing down or stopping on a flight of steps or on a slope, as well as alternatively or in addition when simply standing on the edge or on a sheer place, for example on an outcrop, a rock, an elevated place or a platform. The increase of the resistance is concluded in the right representation of FIG. 3, and by renewed movement of the treated side with the orthopedic joint device it is possible to cancel this increased flexion resistance or the blocking of the joint and to operate the orthopedic joint device in another mode. If the driving via the biosignal is ended at a time when the treated side is not yet sufficiently at rest and the criteria for the activation of a conventional standing function are correspondingly not fulfilled, the resistance is reduced again with the decreasing driving via the biosignal.

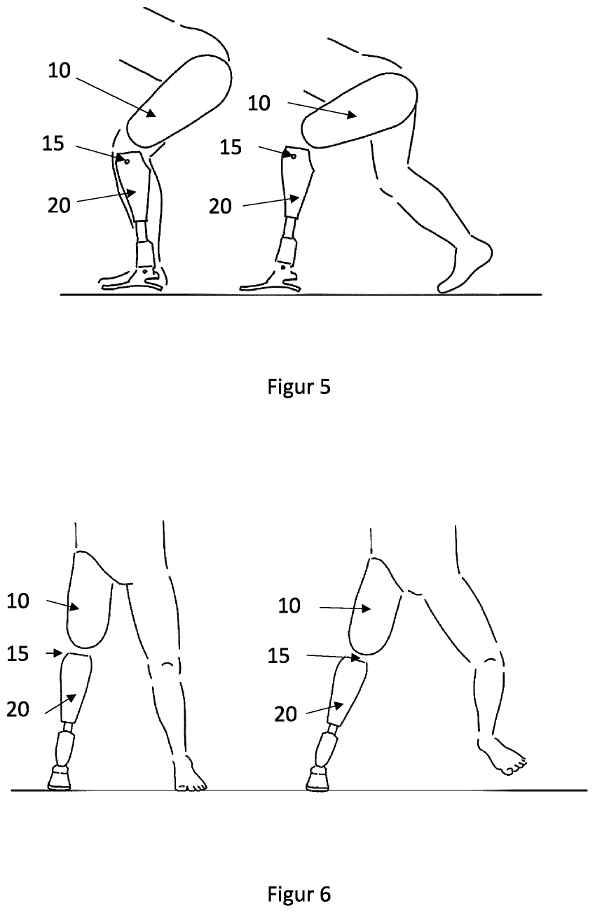

FIG. 4 represents a further possible application, in which a user of the orthopedic joint device is in a bent setting, as represented in the left drawing of FIG. 4. In some situations, the body position is varied without a knee angle being changed, for example by the user bending further forward. In conventional control methods, the standing function already adopted is initially deactivated so that the flexion resistance is reduced. This takes place on the basis of particular sensor values and further parameters, for example only during a knee extension, a rapid movement of the upper part or lower part or full relief. By driving of the actuator via biosignals, the standing function can remain active with an increased resistance when, during the movement of the upper body or of the upper part or lower part, deactivation or driving would take place due to the sensor signals. As represented in the left figure, the user initially stands relaxed in a bent setting. During the position change of the upper body, the orthopedic joint device is driven via a biosignal, for example by a contraction of the thigh musculature, which is recorded in the form of electrical signals via biosignal sensors or lead electrodes and forwarded as biosignals to the control device. The orthopedic joint device remains in the standing function. After the relief of the thigh musculature, a biosignal is no longer applied to the control device but the resistance via the actuator continues to remain high since the user is still in a standing static position. Only when a movement of the treated side takes place is the control function cancelled and the flexion resistance by the actuator reduced.

FIG. 5 shows a further situation, in which a user of the orthopedic joint device is dragging an object (which is not represented). Starting from a bent setting, which is represented in the left drawing, the person steps backward with the untreated healthy side in order to drag an object lying in front of them, or to exert a pull. A conventional standing function would be deactivated in this case because the treated side is rotating back, i.e. the lower part 20 is carrying out a pivoting movement about the ankle joint axis in the backward, posterior direction. If a biosignal is transmitted via the biosignal sensors to the control device, however, it is possible to keep the standing function active deliberately even during the backward movement or backward pivoting of the lower part 20 about an ankle joint axis. The driving via the biosignal may also be maintained in the position of the right representation, for example when the person wishes to ensure that the standing function continues to remain active despite a movement of the components of the joint device. Only when the biosignal is no longer applied, and the person is no longer standing still, is the increased resistance against a flexion movement reduced again. If the person stands still again, however, the driving by the biosignal may be ended in order to enable relaxed standing. The person is thereby relieved cognitively and muscularly. It is likewise possible that the standing function is activated or remains activated by the driving by means of the biosignal even though the activation would normally not be possible in this position on the basis of the other sensor data. If, for example, a condition is that a shank part must be vertical or inclined forward in the walking direction before a standing function with an increased flexion resistance is switched on, this would be an obstacle to providing the setting in the right representation of FIG. 5 with a standing function. With the claimed method, it is possible to overrule the other conditions via the biosignal and increase the resistance against a flexion by the biosignal due to a higher prioritization.

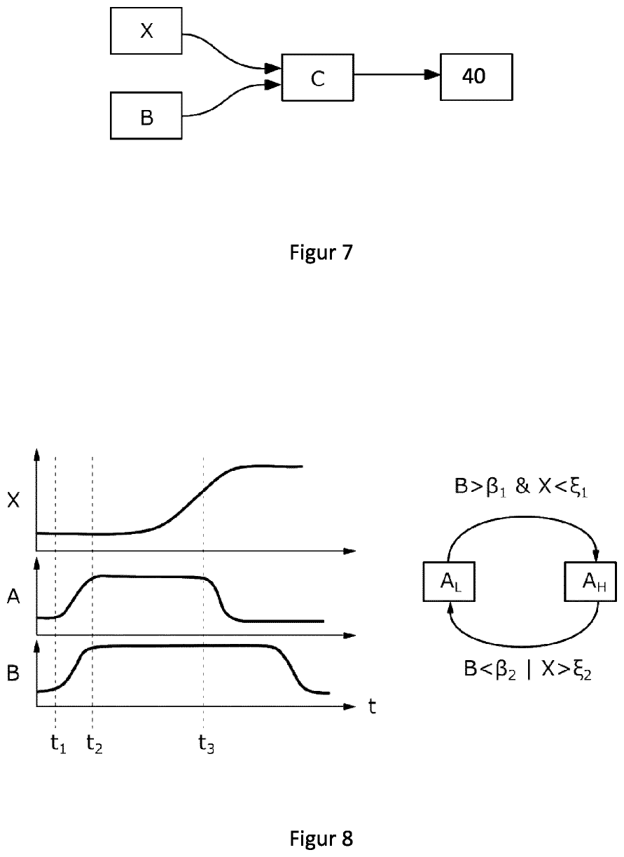

FIG. 6 represents a further situation, in which a lateral movement takes place within the frontal plane. In the exemplary embodiment represented, the person is taking a step to the left while the treated right side remains standing. Conventional controls do not react to movements within the frontal plane, so that a standing function with an increased flexion resistance may also be set up via a biosignal in such situations. An increase of a flexion resistance by a biosignal in the event of a lateral movement is, for example, expedient during a dynamic direction change from a forward movement to a combined forward and sideways movement, in order to be able to push off from the treated side. In the terminal stance phase, a reduction of the flexion resistance of the knee joint may take place based on the other sensors despite driving via the biosignal, in order to initiate a swing phase or to reinforce flexion for the swing phase.

FIG. 7 represents the basic functional scheme of the control. Biosignals B and further items of information X, which are provided by sensors or data sources, such as sensor signals pertaining to state variables, internal states or external data, are processed to a control C which is stored and held in a control device. The control C of the control device then drives the actuator 40. The conventional items of information X are typically kinematic variables such as knee angle, segment angle, ranges of motion, distances, lengths and orientations of a leg tendon, speeds, accelerations and the like, and kinetic variables such as forces, moments, lever arms, force action points and the like. Furthermore, external items of information X may come from an adjustment device, apps, other prosthetic components or a data cloud. The biosignals B are for example data regarding the muscle tension, which are picked up or recorded via electromyographic sensors, ultrasound, magnets, pressure sensors, direct nerve sensors and the like. The control C in the control device generates an input variable for the actuator 40 and therefore a manipulated variable, for example regarding the position of the joint, the position of a valve, the opening or closing of a valve, regarding speeds, moments, current, stiffnesses, dampings, impedance and the like.

FIG. 8 schematically represents an embodiment of the control for an increase of the flexion resistance. The input variables X for sensor values of the sensors 50 according to FIG. 1 are represented in the upper group, the activation or deactivation of the actuator 40 is represented in the middle curve with the activation signal A as a function of time, and the biosignal B is represented as a function of time in the lower curve. The right representation of FIG. 8 indicates two of the actuator states and the conditions therefor. The first state AL denotes a state in which the actuator 40 exhibits a normal resistance behavior, i.e. it adopts a low flexion resistance, or is open. The actuator state AH corresponds to the state with an increased resistance, optionally a blocked actuator which prevents a flexion. For the case that the biosignal B is greater than a limit value β1 and in addition there are values for the sensor values or conventional variables X that lie within a range which would normally lead to an activation of a standing function, which is symbolized by the expression <ξ, a state AL with a low flexion resistance is switched over to a state AH with a high flexion resistance. The control is thus activated only when both the condition, or the conditions, for the sensor values X of the state sensors 50 and the conditions of the biosignals B via the biosignal sensors 60 are fulfilled together. That is to say, the system is for example sufficiently at rest and loaded and there is furthermore driving by the biosignal B. A deactivation, i.e. switching from the state AH to the state AL, takes place when one of the two conditions is not fulfilled. That is to say, when there is no biosignal B of a sufficient size or intensity, or a limit value ξ for the sensor values or state data X is exceeded, the flexion resistance is reduced again. Such an implementation may, for example, be expedient when inadvertent increasing or maintaining of the resistance is particularly undesired, for example in a special mode such as cycling. As expressed in the curves, the orthopedic joint device is at rest at time t1 and there is no biosignal B. The resistance of the actuator 40 is accordingly low, as is shown in curve A. Between times t1 and t2, the biosignal B is increased, for example by tensioning the thigh muscle. Accordingly, the resistance A that is provided by the actuator 40 also increases, in which case the increase or elevation of the resistance may take place proportionally to the muscle signal. At time t3, a sufficient variation of the state of the orthopedic joint device takes place, which is indicated by the rise of the values X. Accordingly, the resistance by the actuator 40 is reduced even though the biosignal B continues to be applied.

In the embodiment according to FIG. 8, the biosignal B is overruled by the sensor values X.

FIG. 9 represents a variant of the control in which a deactivation, i.e. a reduction of the flexion resistance, takes place only when there is no driving by the biosignal B and the other conditions are also no longer fulfilled by the state data X of the joint device. The activation takes place according to upper right representation in FIG. 9 only as a function of the biosignal B, while in the lower right representation the activation of the actuator 40 takes place when neither the biosignal B is sufficient or the sensor values X for state data of the orthopedic joint device lie at an order of magnitude for which an activation of the standing function and an increase of the flexion resistance would take place.

At time t0, the orthopedic joint device in the form of a leg orthosis or of a prosthetic leg is in a forward movement and no driving by the biosignal B is taking place. Between times t0 and t1, the driving A by the biosignal B is increased. Although the leg rotates further forwards, which normally would not lead to an activation of the standing function due to sensor values of the state data X, the resistance is increased via the activation of the actuator 40 according to curve A. By the increase of the flexion resistance, the movement between the upper part and the lower part is reduced and stopped, and the movement of the orthopedic device is braked, which may be read from the decrease of the sensor values of the state data X. The biosignal B is reduced when braking and locking of the joint have taken place, for example by the muscle being relaxed, so that the intensity of the biosignal B is reduced beyond time t2. The increased flexion resistance remains, which is expressed by the constant raised profile of curve A. The person can stand relaxed. If the joint device is then moved again, which may be read from the rising values of the X curve, the resistance against a flexion is reduced, as indicated by the falling curve A.

The upper right representation shows the conditions for switching from the state AL to the state AH, namely when the biosignal B is large enough and has a sufficient quality in order to carry out driving of the actuator with switching to an increased resistance. If the biosignal B is also then reduced or ceases, i.e. the biosignal B<β2, the state of the increased flexion resistance is maintained. The same happens when there is again a biosignal B that is greater than β3, so that there is a sufficient signal quality for driving. Only when a renewed movement of the orthopedic joint device takes place without simultaneous driving via the biosignal, i.e. the state values X>ξi, that is to say they lie outside a range that would normally lead to an activation of a standing function with an increased flexion resistance, is the flexion resistance reduced and the state AL adopted. In the lower right representation, an increase of the flexion resistance is carried out when one of the two conditions are fulfilled, i.e. either the biosignal B is greater than a limit value β that would justify driving of the actuator or when the sensor values for the state variables lie within a range that would normally lead to an activation of a standing function and an increase of the flexion resistance. Unlocking, or a reduction of the flexion resistance, takes place only when the sensor values for the state data X for the joint device lie outside a range that would normally lead to an increase of the flexion resistance, for example when the entire prosthesis is lifted and pivoted and driving does not simultaneously take place via the biosignal.

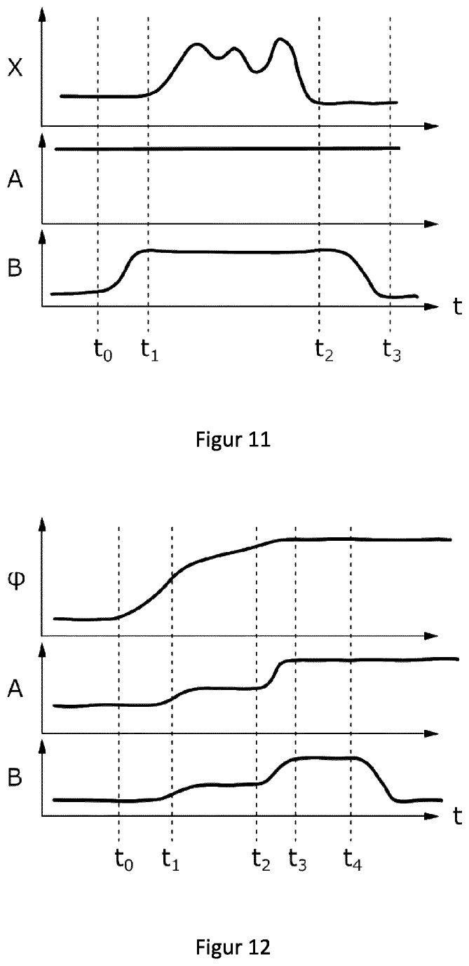

FIG. 10 represents the control for a special mode. The special mode may for example be a special function for riding a bicycle, in which periodically recurring movements are carried out. Within such a mode, it is then possible that an increase of the resistance takes place only by the biosignal B. At a time t1, the biosignal B is increased up to a time t2, for example by an increasing muscle contraction. The biosignal B remains at a higher level than at time t1. Together with the increase of the biosignal B, an activation of the actuator takes place with an increase of the flexion resistance, which is shown in the middle curve A. After the reduction and maintaining of the muscle tension at an increased level above a threshold value beyond time t2 until time t4, the flexion resistance also remains at an increased level. The increase of the resistance may also be triggered by the quality of the biosignal B, for example by a significant impulse or via a duration for which the biosignal B is maintained. The conditions for the quality of the biosignal B serve to prevent an inadvertent increase of the flexion resistance. Only when a biosignal B again occurs above the level at time t4, and possibly decreases again, is the resistance reduced. Accordingly, the movements also vary, which may be recognized by the values of the state data X. These data may be used to activate another control strategy or another control mode. For example, these switchings may be utilized when riding a bicycle in order to rise up and stand on the pedals. The pedal movements or vibrations should not on their own be decisive for the activation or deactivation of the increased resistance.

FIG. 11 shows a variant of the control according to FIG. 9, in which the locked setting or the increased flexion resistance is indicated by the high values of curve A. No driving by the biosignal B takes place. The user of the orthopedic joint device is standing for example relaxed in a bent setting, and the biosignal B may now anticipate a situation in which the resistance of a conventional control would be reduced, for example when the state data X would entail unlocking or reduction of the flexion resistance. If, with an already increased resistance, driving via the biosignal B takes place at time t0 and a movement of the orthopedic joint device is carried out only after this, which is represented by the rising and falling curve in the period between t1 and t2 of the upper curve X, the resistance remains high during the movement in this period, which is expressed by the constant profile of curve A. When the movement is ended at time t2, the biosignal B may also be reduced, which takes place in the period between times t2 and t3. The conditions for a reduction of the flexion resistance on the basis of the sensor values of the state data X are, beyond time t2, also in a range in which no unlocking would take place, so that the locked setting or the standing function still exists in an orthopedic joint device of the lower extremity. The flexion resistance continues to remain high, and unlocking does not take place.

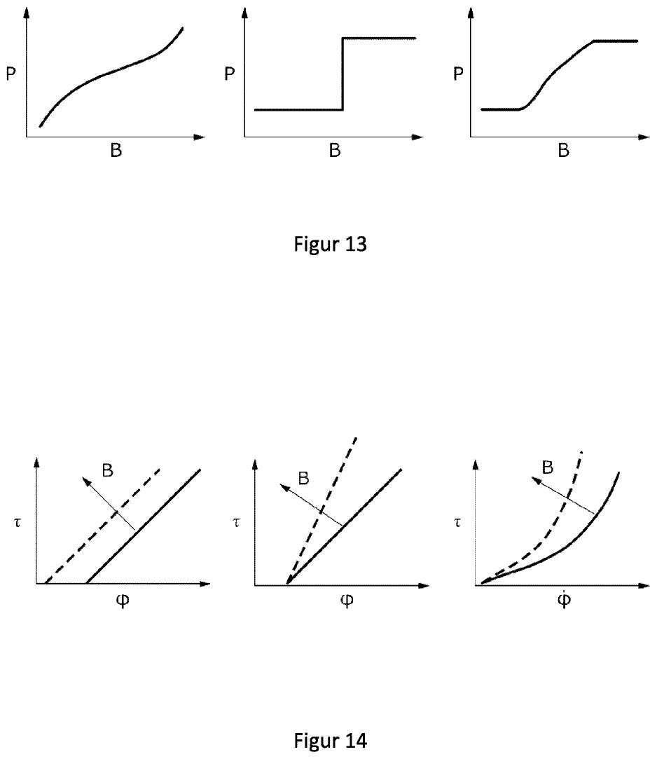

FIG. 12 shows a further control variant, in which a flexion movement through the knee angle ok is represented. The knee angle ok is represented in the upper diagram. At the start of the control, there is a normal or low resistance behavior for the actuator A. No biosignal B is applied, and the knee joint is in a sufficiently extended position. If the orthopedic joint device, for example in the form of a prosthesis as in FIG. 3, is placed on the next step down and the artificial knee joint flexes under load in a controlled way, which happens in the period between t0 and t1, in order to reduce the flexion beyond time t1 a first biosignal B at an increased level, for example a medium-strength stimulation of the musculature, is supplied to the control in order to achieve a medium resistance. This resistance is built up beyond time t1 and maintained until time t2. This allows controlled slowing of the flexion movement without ending it abruptly. At time t2, the movement is intended to be stopped. For this purpose, the biosignal B is increased in the period between times t2 and t3, for example by a maximum contraction of the corresponding muscle. The resistance A that is provided by the actuator is thereby maximally increased and the variation of the knee angle φk is slowed, so that the movement can come to a stop at time t3. The biosignal B may be ended, i.e. the muscle tension or muscle stimulation may be reduced, at time t4 when the knee flexion has fully come to a stop and a standing function is installed. The resistance by the actuator nevertheless remains high until a corresponding cancellation signal is supplied to the control.

In addition or as an alternative to a control via simple threshold values, continuous transitions between the resistances may be produced. For example, criteria may be partially fulfilled and lead to a partial increase or reduction of the resistance.

FIG. 13 represents different relationships between the biosignal B and the resistance parameter P. The resistance behavior of the actuator is increased by the driving via the biosignal B, in which case different parameters P of the resistance behavior may be influenced. This is explained in more detail in FIG. 14. The type of influencing may be continuous, or proportional, that is to say an increased resistance is provided in the event of an increased activation of the musculature or of the biosignal B, which is shown in the left representation of FIG. 13. The relationship between the biosignal B and the resistance parameter P need not be linear. As an alternative, the adaptation may take place in a binary fashion, that is to say a fixed increase of the resistance is carried out beyond a particular threshold value or a plurality of particular threshold values of the biosignal B. This is represented in the middle representation of FIG. 13. Likewise, the adaptation of the resistance parameters P may have saturation ranges so that a further increase of the driving by the biosignal B leads to no further increase of the resistance. This is represented in the right representation of FIG. 13. The different types of adaptation may also be combined with one another and the different types of adaptation may likewise be used differently in different situations; for example, binary switching may take place when riding a bicycle while proportional adaptation takes place when standing.

FIG. 14 represents different types of resistance increase with reference to the example of a resilient behavior and a damping behavior, a resistance moment T being plotted as a function of the knee angle pi. The resistance moment T is applied against a movement or with a movement, in order to cause influencing of the pivoting movement. In the case of a resilient behavior, an equilibrium position may be varied by the biosignal B. When a moment that extends the knee is generated, which is achieved by flexion against a spring behavior, the equilibrium position of the spring may be shifted toward a smaller knee angle when driving via the biosignal B. For the same knee angle φi, a higher knee extension moment is then applied. The spring stiffness may likewise be varied, which is represented in the central representation of FIG. 14. In the case of a damping behavior, the damping coefficient may be increased by the driving via the biosignal B. In the case of nonlinear characteristics, correspondingly more coefficients are to be varied, or the corresponding characteristic curve is shifted toward higher moments. The increase and/or reduction of a resistance may therefore also be the increase and/or reduction of a stiffness, of an equilibrium point, of a damping and/or the like. Other parameters of such characteristics, for example the progressivity, may also be adapted in the scope of a resistance variation. Several characteristics may also be combined, for example a resilient behavior and damping behavior. As an alternative or in addition to the knee angle, φ may also be another pivoting degree of freedom of the orthopedic device which is countered by a resistance or the pivoting movement of which is influenced by an actuator. Alternatively, it may however also involve another state of the orthopedic device that is recorded via the sensors, for instance the absolute angle of the upper or lower part or the load. Naturally, the moment, or the force applied by the actuator, may also be influenced directly by the biosignal against or with the pivoting, particularly in a proportional relationship, which corresponds to a reduction or increase of the resistance.

FIG. 15 represents a further working situation of an orthopedic joint device, namely repositioning of the treated leg. In the initial setting represented on the left, the user is in a slightly bent posture. A standing function of the orthopedic device is active and provides corresponding resistances against a knee flexion and against a dorsiflexion. For the repositioning, the treated side is relieved and the leg is placed forward, in this case on an edge of a step as represented in the right drawing, and then loaded again. In the event of relief or pivoting, the usual standing function would be deactivated and the resistances in the knee or ankle would be reduced. Adopting a position as represented on the right and loading the prosthesis would be possible only with great difficulty since the position is very unstable and both the knee joint and the ankle joint would experience a strongly flexing moment. If, however, a biosignal is transmitted via the biosignal sensors, for example the electrodes of the control device, the standing function may deliberately be kept active even during the relief and repositioning, in this case essentially pivoting about the hip. After the repositioning and loading, the driving via the biosignal may cease again from this quasistatic situation, the standing function nevertheless remaining active. This is possible because the reduction of the resistances takes place based on sensor data, in particular sensor data pertaining to the state of the orthopedic device. In the case of repositioning as represented, it is advantageous to increase not only the resistance against a flexion but also the resistance against an extension in the knee and/or against a plantarflexion in the joint based on the biosignal, optionally to the extent of blocking. If the biosignal ceases after the repositioning, the resistance against an extension and/or a plantarflexion may be reduced again.

1. A method for controlling an orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor and comprising at least one biosignal sensor, which records a muscle activity or a stimulation of at least one muscle as a biosignal and transmits it to the control device, the actuator being activated, deactivated or modulated on the basis of the biosignal or the biosignals, characterized in that the resistance to the pivoting movement by the actuator against a flexion is increased on the basis of at least one biosignal, and the increased resistance against a flexion is subsequently reduced on the basis of sensor values.

2. The method as claimed in claim 1, characterized in that the increased flexion resistance is reduced only on the basis of sensor values or on the basis of a combination of sensor values and at least one biosignal.

3. The method as claimed in claim 1, characterized in that the sensors detect a movement of the upper part or of the lower part absolutely or relative to one another.

4. The method as claimed in claim 1, characterized in that the increased flexion resistance is maintained, independently of a variation of the biosignal, until at least one sensor signal due to a movement, load and/or state change of a defined size is detected by the at least one sensor.

5. The method as claimed in claim 1, characterized in that the biosignal triggers the increase of the flexion resistance independently of the position, load and/or movement of the joint device or of the upper part or of the lower part.

6. The method as claimed in claim 1, characterized in that the biosignal triggers the increase of the flexion resistance only if no position, movement or state change of the upper part and/or lower part, or one that lies below a limit value, is detected by the at least one sensor and/or a load above a limit value, in particular an axial load against the direction of gravity, of the upper part and/or lower part is detected.

7. The method as claimed in claim 1, characterized in that the sensor values must exceed or fall below a threshold value before a reduction of the flexion resistance is initiated.

8. The method as claimed in claim 1, characterized in that the flexion resistance is reduced despite stimulation via the at least one biosignal on the basis of the at least one sensor signal of the at least one sensor due to a movement, load and/or state change of a defined size.

9. The method as claimed in claim 1, characterized in that the extent of the increase and/or of the flexion resistance is varied on the basis of the position, the movement, the load, the state change, the mode and/or the movement phase.

10. The method as claimed in claim 1, characterized in that the at least one biosignal is a myoelectrical signal, which is recorded in particular by implanted electrodes or electrodes fitted on the surface.

11. The method as claimed in claim 1, characterized in that the flexion resistance is increased to the extent of blocking.

12. The method as claimed in claim 1, characterized in that the flexion resistance is reduced on the basis of at least one sensor value to a level below the initial resistance before increasing the flexion resistance, particularly in a terminal stance phase.

13. The method as claimed in claim 1, characterized in that, in at least one mode or at least one movement phase, the stimulation of a muscle or a muscle group is recorded and employed for the control.

14. The method as claimed in claim 1, characterized in that, in at least one mode or at least one movement phase, the cocontraction of at least two muscles or muscle groups is recorded and employed for the control.

15. The method as claimed in claim 1, characterized in that the flexion resistance is increased continuously with increased activation of the musculature and/or reduced with reduced activation and/or the flexion resistance is controlled digitally by the activation of the musculature.

16. An orthopedic joint device comprising an upper part and a lower part, which are mounted articulated to one another so that they can pivot about a pivot axis, comprising an actuator, which is coupled to the upper part and to the lower part and influences a pivoting movement of the upper part relative to the lower part, the actuator being coupled to a control device, which is coupled to at least one sensor for recording state data of the joint device and activates, deactivates or modulates the actuator on the basis of sensor values of the at least one sensor and comprising at least one biosignal sensor, which records biosignals and transmits them to the control device, the actuator being activated, deactivated or modulated on the basis of the biosignals, characterized in that the control device is adapted to increase the influencing by the actuator against a flexion on the basis of a biosignal and to reduce the increased flexion resistance on the basis of sensor values.

17. The orthopedic joint device as claimed in claim 16, characterized in that it is formed as a joint device of the lower extremity, in particular an orthotic hip joint, prosthetic hip joint, orthotic knee joint, prosthetic knee joint, orthotic ankle joint, prosthetic ankle joint or as a joint device of the upper extremity, in particular as an artificial elbow joint or artificial shoulder joint an orthosis, prosthesis or of an exoskeleton.

18. The orthopedic joint device as claimed in claim 16, characterized in that the at least one sensor is arranged on the upper part and/or on the lower part.
